(19) United States
(12) Patent Application Publication
(10) Pub. No.: US 2012/0196248 A1
(43) Pub. Date: Aug. 2, 2012

(54) ARTIFICIAL TOOTH ROOT

(75) Inventor: Gue-Wha HUANG, Miaoli County (TW)

(73) Assignee: National Chiao Tung University, Hsinchu (TW)

(21) Appl. No.: 13/355,054

(22) Filed: Jan. 20, 2012

(30) Foreign Application Priority Data
Jan. 27, 2011 (TW) 100103077

(51) Int. Cl. A61C 8/00; B82Y 5/00

(52) U.S. Cl. 433/173; 977/742; 977/773

(57) ABSTRACT

An artificial tooth root includes a root body adapted to be embedded in a jawbone, and a nano-structure layer having a base portion disposed on the root body, and a plurality of separated nanodots that are formed on the base portion and that protrude from the base portion oppositely of the root body.

### CROSS-REFERENCE TO RELATED APPLICATION

This application claims priority of Taiwanese application no. 100103077, filed on Jan. 27, 2011.

### BACKGROUND OF THE INVENTION

1. Field of the Invention

This invention relates to an artificial tooth root, more particularly to an artificial tooth root that comprises a nano-structure layer.

2. Description of the Related Art

An artificial tooth is employed to replace missing natural tooth. In order to install the artificial tooth, natural teeth adjacent to the missing tooth have to be partially removed so as to firmly fix the artificial tooth. Thus, the natural teeth are inevitably damaged.

In order to overcome the aforesaid drawback, an artificial tooth that has an artificial tooth root embedded in a user' s jawbone has been developed. Referring to FIG. 1, the artificial tooth includes an artificial tooth root 11 formed with a screw thread 12 and screwed into a user's jawbone 13, a connector 15 disposed on the tooth root 11, and an artificial tooth crown 14 mounted on the connector 15. Bone cells are usually grown on a surface of the artificial tooth root 11. After embedding the artificial tooth root 11 in the jawbone 13 for a period of time, the bone cells will be integrated with the jawbone 13, thereby firmly bonding together the artificial tooth root 11 and the jawbone 13. However, before the bone cells are integrated with the jawbone 13, the artificial tooth root 11 is held in the jawbone 13 primarily via on the screw thread 12. Besides, the bone cells are difficult to attach to and grow on the surface of the artificial tooth root 11, and thus, a long proliferation time for the bone cells is required.

To overcome the problem of long proliferation time, an artificial tooth root 11 having a layer of osteoconductive material coated thereon so as to facilitate the attachment and growth of the bone cells has been developed. However, the osteoconductive material is likely to detach and degrade with time, so that the connection between the artificial tooth root 11 and the jawbone 13 becomes weak, and the artificial tooth root 11 may be loosened due to occlusal force.

### SUMMARY OF THE INVENTION

Therefore, the object of the present invention is to provide an artificial tooth root that can overcome the above drawbacks of the prior art.

The artificial tooth root according to the present invention comprises a root body adapted to be embedded in a jawbone, and a nano-structure layer having a base portion disposed on the root body, and a plurality of separated nanodots that are formed on the base portion and that protrude from the base portion oppositely of the root body.

### BRIEF DESCRIPTION OF THE DRAWINGS

Other features and advantages of the present invention will become apparent in the following detailed description of the preferred embodiments of this invention, with reference to the accompanying drawings, in which:

FIG. 1 is a schematic view of a conventional artificial tooth;

FIG. 2 is a fragmentary schematic view of the preferred embodiment of an artificial tooth root of the present invention;

FIG. 3 is a schematic view of the preferred embodiment of the artificial tooth root;

FIG. 4 is a fragmentary schematic view illustrating a tantalum nitride film and an aluminum film formed on a root body of the preferred embodiment;

FIG. 5 is a fragmentary schematic view illustrating a plurality of holes formed in an aluminum oxide film after the aluminum film shown in FIG. 4 is subjected to anodic oxidation;

FIG. 6 is a scanning electron microscopic photograph showing a nano-structure layer of the artificial tooth root shown in FIG. 2;

FIG. 7 is a diagram showing the results of cell adhesion percentage of MG63 cells grown on artificial tooth roots A to C of Examples 1 to 3 and artificial tooth roots D and E of Comparative Examples 1 and 2;

FIG. 8 is a diagram showing the results of cell proliferation percentage of MG63 cells grown on the artificial tooth roots A to C of Examples 1 to 3 and the artificial tooth roots D and E of Comparative Examples 1 and 2;

FIG. 9 is a diagram showing the results of cell apoptosis percentage of MG63 cells grown on the artificial tooth roots A to C of Examples 1 to 3 and the artificial tooth roots D and E of Comparative Examples 1 and 2; and

FIG. 10 is a diagram showing the results of cell mineralization percentage of MG63 cells grown on the artificial tooth roots A to C of Examples 1 to 3 and the artificial tooth roots D and E of Comparative Examples 1 and 2.

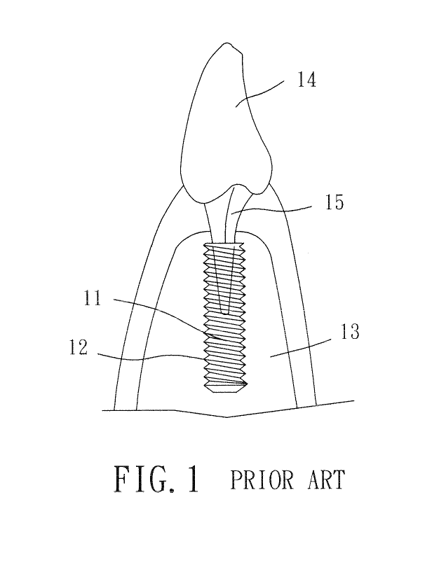

### DETAILED DESCRIPTION OF THE PREFERRED EMBODIMENTS

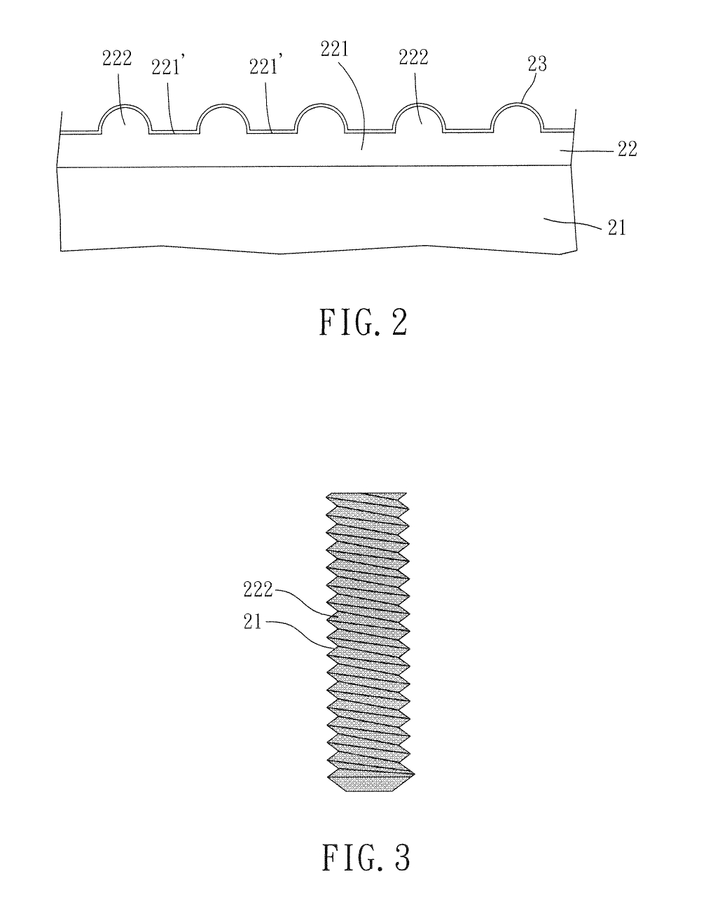

FIGS. 2 and 3 shows the preferred embodiment of an artificial tooth root according to the present invention that comprises a root body 21 adapted to be embedded in a jawbone, a nano-structure layer 22 having abase portion 221 that is disposed on the root body 21, and a plurality of separated nanodots 222 that are formed on the base portion 221 and that protrude from the base portion 221 oppositely of the root body 21, and an optional cell-attachment improving layer 23 which is formed on the nanodots 222 and parts of the base portion 221′ not covered by the nanodots 222 (hereinafter referred to as the exposed base portion).

The root body 21 is usually has a column shape with a smooth surface or roughed surface on which the nano-structure layer 22 is formed. Since the nano-structure layer 22 has a relatively small thickness, the artificial tooth root has an appearance shape the same as that of the root body 21. Preferably, the root body 21 of this invention has a roughed surface. In an embodiment of this invention, the roughed surface is a screw-shaped surface on which the nano-structure layer 22 is formed (see FIG. 3)

The base portion 221 has a thickness ranging from 10 nm to 200 nm. Each of the nanodots 222 has an average size ranging from 10 nm to 90 nm, preferably from 35 nm to 65 nm, and an average height ranging from 10 nm to 100 nm. The nano-structure layer 22 is made from a material selected from the group consisting of metal, metal oxides, metal nitrides, and combinations thereof. Preferably, the nano-structure layer 22 is made from a material selected from the group consisting of tantalum, tantalum oxides, tantalum nitrides, and combinations thereof.

The cell-attachment improving layer 23 is made from platinum, and is formed on the exposed base portion 221′ and the nanodots 222 of the nano-structure layer 22 by a sputtering method. The cell-attachment improving layer 23 has a thickness larger than 0 nm and less than 10 nm, and preferably, is 6 nm. The cell-attachment improving layer 23 covers the nano-structure layer 22 so as to reduce metal property and surface energy of the nano-structure layer 22, thereby eliminating the effect of the material property of the nano-structure layer 22 on the bone cells.

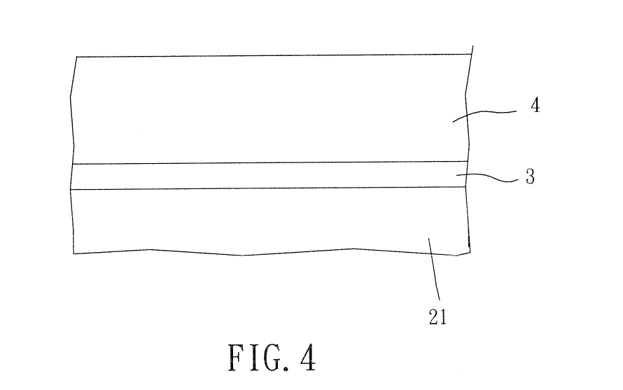

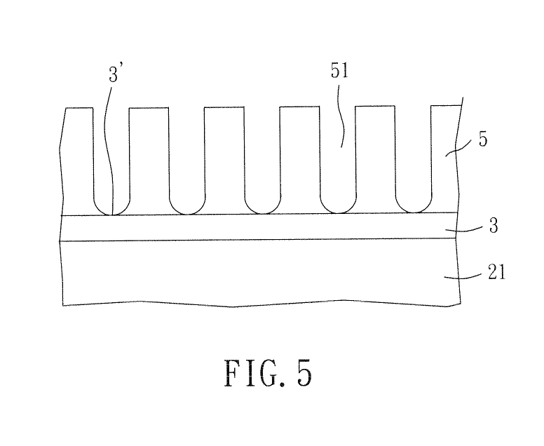

Referring to FIG. 4, the preferred embodiment of the artificial tooth root of this invention is prepared according to the following steps. First, the tantalum nitride film 3 is deposited on the root body 21. In an embodiment of this invention, the root body 21 is a silicon wafer. The tantalum nitride film 3 is used as an oxidation barrier layer, and has a good biocompatibility because of the inert property of tantalum. Then, the aluminum film 4 is deposited on the tantalum nitride film 3.

The root body 21 is subsequently subjected to anodic oxidation. The aluminum film 4 is anodically oxidized, thereby forming an aluminum oxide film 5 with the plurality of holes 51 (see FIG. 5). Parts of the tantalum nitride film 3 are exposed from the holes 51 in the aluminum oxide film 5. Next, the exposed tantalum nitride film 3′ is subjected to oxidation to form the nanodots 222. The aluminum oxide film 5 is then removed using phosphoric acid. Thus, the nano-structure layer 22 is formed on the root body 21, in which the tantalum nitride film 3 is the base portion 221 of the nano-structure layer 22. The exposed base portion 221′ and the nanodots 222 are coated with the cell-attachment improving layer 23 by sputtering.

### Example 1

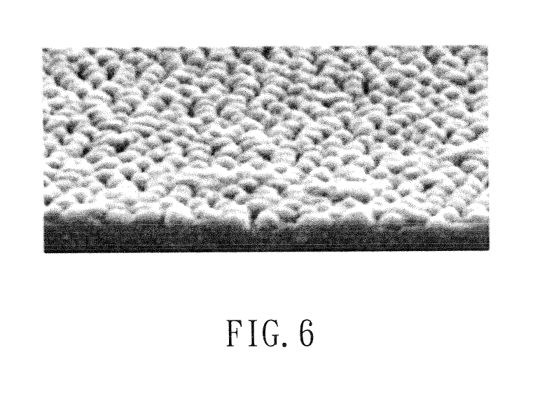

A root body 21 made of a silicon wafer was prepared, and a tantalum nitride film 3 with a thickness of 200 nm was deposited on the root body 21. Then, an aluminum film 4 with a thickness of 400 nm was deposited on the tantalum nitride film 3. The aluminum film 4 was subjected to anodic oxidation using a voltage of 5 V in 1.8 M of sulfuric acid solution used as a plating solution, thereby forming an aluminum oxide film 5 with a plurality of holes 51 with an average diameter of 10 nm. Parts of the tantalum nitride film 3 were thus exposed from the holes 51. Next, the exposed tantalum nitride film 3′ was oxidized to form nanodots 222 with an average diameter of 10 nm and an average height of 10 nm. The aluminum oxide film 5 was then removed using a phosphoric acid solution (5 g of phosphoric acid in 100 ml water). The nanodots 222 and parts of the tantalum nitride film 3 not covered by the nanodots 222, were coated with a cell-attachment improving layer 23 with a thickness of 6 nm by a sputtering method, thereby obtaining an artificial tooth root A (see FIG. 6).

### Example 2

The method for preparing the artificial tooth root of Example 2 (i.e., artificial tooth root B) was similar to that of Example 1, except that the plating solution was 0.3 M oxalic acid solution and the voltage used in anodic oxidation of the aluminum film 4 was 25 V. Since the aluminum oxide film 5 thus formed had a plurality of holes 51 with an average diameter of 50 nm, the nanodots 222 thus formed had an average diameter of 50 nm and an average height of 50 nm.

### Example 3

The method for preparing the artificial tooth root of Example 3 (i.e., artificial tooth root C) was similar to that of Example 1, except that the plating solution was 0.3 M oxalic acid solution and the voltage used in anodic oxidation of the aluminum film 4 was 100 V. Since the aluminum oxide film 5 thus formed had a plurality of holes 51 with an average diameter of 90 nm, the nanodots 222 thus formed had an average diameter of 90 nm and an average height of 100 nm.

### Comparative Example 1

The method for preparing the artificial tooth root of Comparative Example 1 (i.e., artificial tooth root D) was similar to that of Example 1, except that the plating solution was 5% w/v phosphoric acid solution (5 g of phosphoric acid in 100 ml water) and the voltage used in anodic oxidation of the aluminum film 4 was 100 V. Since the aluminum oxide film 5 thus formed had a plurality of holes 51 with an average diameter of 200 nm, the nanodots 222 thus formed had an average diameter of 200 nm and an average height of 200 nm.

### Comparative Example 2

The method for preparing the artificial tooth root of Comparative Example 2 (i.e., artificial tooth root E) was similar to that of Example 1, except that the anodic oxidation of the aluminum film 4 and the oxidation of the tantalum nitride film 3 were dispensed with, thereby obtaining a flat tantalum nitride film 3 on the root body 21, i.e., no nanodots were formed.

### (1) Test for Cell Adhesion

A predetermined amount of MG63 human osteosarcoma cells (6×10⁴ cells) purchased from “Food Industry Research and Development Institute” was attached to each of the artificial tooth roots A to E. The artificial tooth roots A to E attached with MG63 cells were disposed culture in a culture medium containing 90% α-MEM (Hyclone) and 10% fetal bovine serum (Dulbecco' s Modified Eagle' s Medium, GIBCO) in a 6 cm plate, followed by incubation in an incubator containing 5% CO2 at 37° C. for 3 days. The MG63 cells were fixed with 4% formaldehyde for 15 minutes, rinsed with phosphate-buffered saline (PBS) once, and permeabilized with Triton X-100. The cells were subjected to fluorescence staining using anti-vinculin antibody and phalloidin, observed using a laser scanning confocal microscope (Leica TCS SPE), and subjected to image analysis using Image J software to obtain fluorescence intensity, thereby determining the cell adhesion ability.

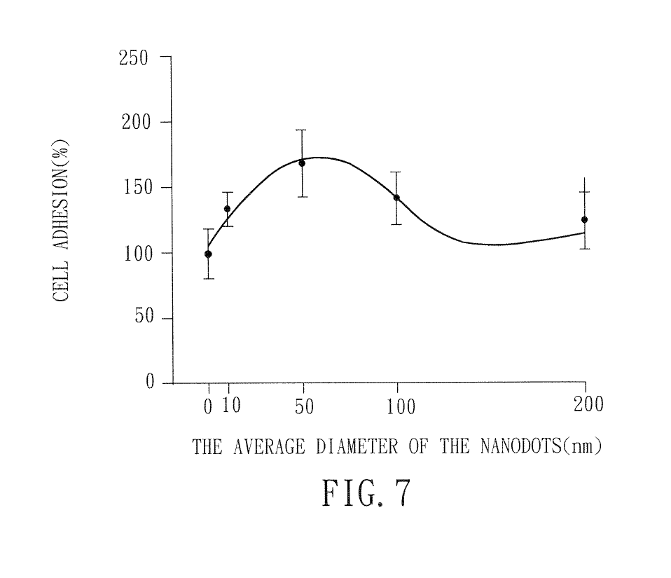

Referring to FIG. 7, the cell adhesion percentage for the artificial tooth root E was designated as 100%, and the cell adhesion percentages for the artificial tooth roots A to D were calculated based on that of the artificial tooth root E. The artificial tooth roots A to C, which respectively had the nanodots with an average diameter ranging from 10 nm to 100 nm, have superior cell adhesion percentages. Particularly, the cell adhesion percentage for the artificial tooth root B, which had the nanodots with an average diameter of 50 nm, is higher than 150%.

### (2) Test for Cell Proliferation

A predetermined amount of MG63 human osteosarcoma cells (4×10⁴ cells) was attached to each of the artificial tooth roots A to E, and was cultivated under the same conditions as set forth in the paragraph of “(1) Test for cell adhesion”. The MG63 cells were dehydrated, and the number of the MG63 cell per unit area was counted using a field emission scanning electronic microscope (JEOL JEM 6700F)

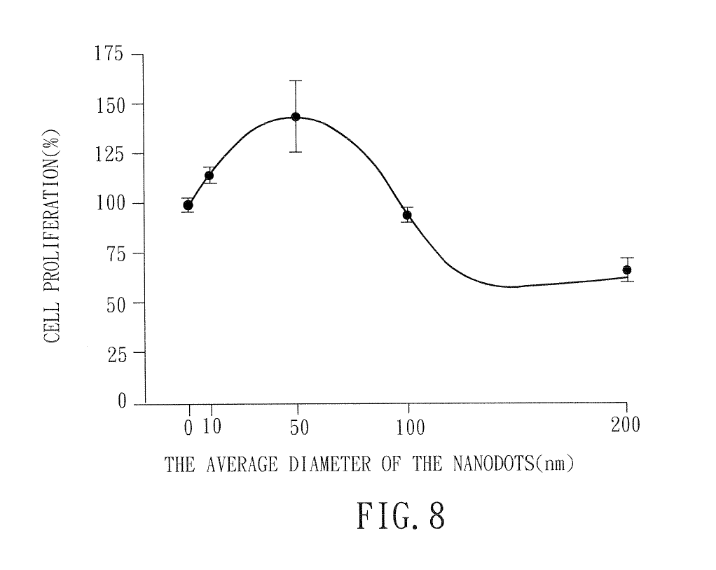

Referring to FIG. 8, the cell proliferation percentage for the artificial tooth root B was designated as 100%, and the cell proliferation percentages for the artificial tooth roots A to D were calculated based on that of the artificial tooth root B. The cell proliferation percentages for the artificial tooth roots A and B are higher than that of the artificial tooth root E. The cell proliferation percentage for the artificial tooth root D is relatively lower than that of the artificial tooth root E. Particularly, the cell proliferation percentage for artificial tooth root B is up to 150%.

### (3) Test for Cell Apoptosis

A predetermined amount of MG63 human osteosarcoma cells (4×10⁴ cells) was attached to each of the artificial tooth roots A to E and was cultivated under the same conditions as set forth in the paragraph of “(1) Test for cell adhesion”. The MG63 cells were dehydrated, and the number of the alive MG63 cell per unit area and the number of the apoptotic MG63 cell per unit area were counted using a field emission scanning electronic microscope (JEOLJEM 6700F). The cell apoptosis percentage was calculated through dividing the number of the apoptotic MG63 cell by the total number of the MG63 cell.

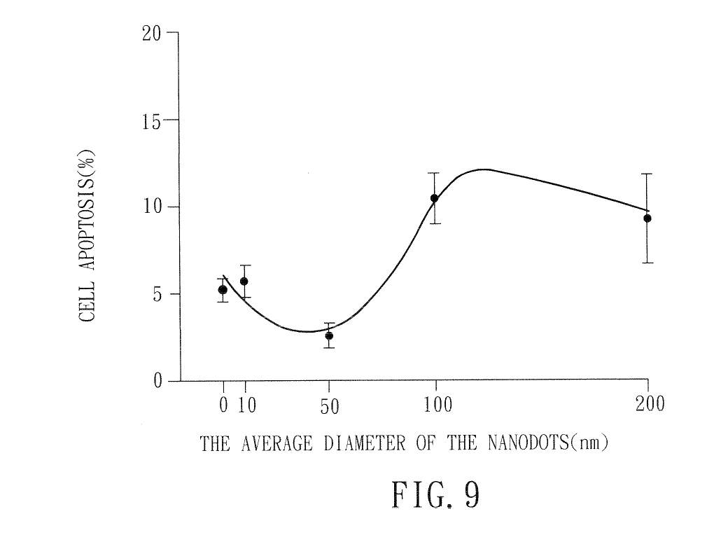

Referring to FIG. 9, in each of the artificial tooth roots A to F., the cell apoptosis percentage is lower than about 10%. Particularly, from the curve plotted in FIG. 9, when the nanodot had an average diameter ranging from 10 to 75 nm, the cell apoptosis percentage can be reduced to less than 5%. Preferably, the average diameter of the nanodot ranges from 35 to 65nm.

### (4) Test for Cell Mineralization

A predetermined amount of MG63 human osteosarcoma cells (4×10⁴ cells) was attached to each of the artificial tooth roots A to E and was cultivated under the same conditions as set forth in the paragraph of “(1) Test for cell adhesion”. The culture medium, was removed. The MG63 cells were rinsed twice with a 1× phosphate buffer, disposed in a solution containing 5% of silver ions, and exposed to sunlight for one hour, so that the MG63 cells became black. Next, the MG63 cells were washed three times with deionized water, stained with nuclear fast red for 5 minutes, and again washed to remove excess nuclear fast red. The stained MG63 cells were observed using microscopy, and were subjected to image analysis using Image J software to determine the level of the cell mineralization.

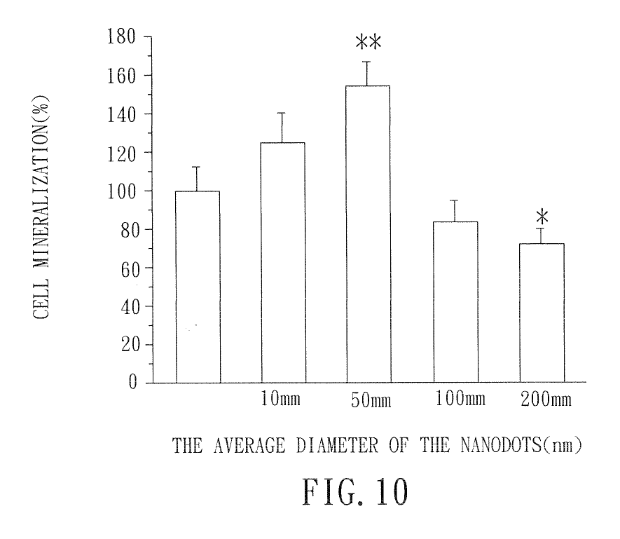

Referring to FIG. 10, the cell mineralization percentage for the artificial tooth root E was designated as 100%, and the cell mineralization percentages for the artificial tooth roots A to D were calculated based on that of the artificial tooth root E. The cell mineralization percentages for the artificial tooth roots A and B are higher than that of the artificial tooth root E. Particularly, the cell mineralization percentage for artificial tooth root B is up to 150%.

According to the test results mentioned above, when the nanodots have an average diameter ranging from 10 nm to 90 nm, the bone cells grown on the artificial tooth root exhibit good cell adhesion and cell proliferation properties. Further, when the average diameter of the nanodots is between 35 nm to 65 nm, the results for cell adhesion, cell proliferation, cell apoptosis and cell mineralization are better.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. An artificial tooth root, comprising:
a root body adapted to be embedded in a jawbone; and
a nano-structure layer having a base portion disposed on said root body, and a plurality of separated nanodots that are formed on said base portion and that protrude from said base portion oppositely of said root body.

2. The artificial tooth root of claim 1, wherein each of said nanodots has an average size ranging from 10 nm to 90 nm.

3. The artificial tooth root of claim 1, wherein each of said nanodots has an average size ranging from 35 nm to 65 nm.

4. The artificial tooth root of claim 3, wherein each of said nanodots has an average height ranging from 10 nm to 100 nm.

5. The artificial tooth root of claim 4, wherein said base portion has a thickness ranging from 10 nm to 200 nm.

6. The artificial tooth root of claim 1, wherein said nano-structure layer is made from a material selected from the group consisting of metal, metal oxides, metal nitrides, and combinations thereof.

7. The artificial tooth root of claim 1, further comprising a cell-attachment improving layer formed on said base portion and said nanodots.

8. The artificial tooth root of claim 7, wherein said cell-attachment improving layer has a thickness larger than 0 nm and less than 10 nm.

9. The artificial tooth root of claim 8, wherein said cell-attachment improving layer is made from platinum.
